(19) United States
(12) Patent Application Publication
(10) Pub. No.: US 2023/0363804 A1
(43) Pub. Date: Nov. 16, 2023

(54) CONNECTOR FOR SPINAL CORRECTION

(71) Applicant: UMC UTRECHT HOLDING B.V., UTRECHT (NL)

(72) Inventors: Moyo C. KRUYT, UTRECHT (NL); René M. CASTELEIN, UTRECHT (NL)

(73) Assignee: UMC UTRECHT HOLDING B.V., UTRECHT (NL)

(21) Appl. No.: 18/029,562

(22) PCT Filed: Oct. 8, 2021
(86) PCT No.: PCT/NL2021/050611

§ 371 Date: Mar. 30, 2023

(30) Foreign Application Priority Data
Oct. 8, 2020 (NL) 2026643

(51) Int. Cl. A61B 17/70

(52) U.S. Cl. CPC A61B 17/705; A61B 17/7079

(57) ABSTRACT

A connector for slidably connecting an anchoring device to a rod in a spinal correction system, comprising a body, a coupling mechanism for coupling the body to the anchoring device, a guiding mechanism arranged to guide the rod in a sliding movement along a longitudinal direction of the rod, wherein said guiding mechanism is further arranged to prevent any movement, with respect to the rod, other than a translation along the longitudinal direction of the rod and a rotation around said longitudinal direction of the rod.

The present invention relates to a connector for connecting an anchoring device to a rod in a spinal correction device, a connector assembly comprising the said connector, a spinal correction system comprising the said connector and a method for spinal correction using said connector.

Spine surgeons can use many types of systems for spinal fixation. A known growth-guiding system for instance comprises a rod coupled to one end of the spine, a connector slidably coupled to said rod which connects to an anchoring device on the other end of the spine. The connector then allows relative movement of the rod and thus between the connection point at the lower end of the spine and the anchoring point. It is also known to use helical springs positioned around the rod to allows continuous distraction forces, which stimulates spinal growth.

An object of the invention, next to other objects, is to provide an improved connector for slidably connecting an anchoring device to a rod in a spinal correction system.

This object, next to other objects, is met by a connector according to claim 1. Specifically, this is met by a connector for slidably connecting an anchoring device to a rod in a spinal correction system, comprising a body, a coupling mechanism for coupling the body to the anchoring device, a guiding mechanism arranged to guide the rod in a sliding movement along a longitudinal direction of the rod, wherein said guiding mechanism is further arranged to prevent any movement, with respect to the rod, other than a translation along the longitudinal direction of the rod and a rotation around said longitudinal direction of the rod. Said guiding mechanism is thus preferably further arranged to prevent any movement of the body other than a movement with respect to said longitudinal direction of the rod. In this way, in particular movement of the rod in the sagittal plane is prevented, while keeping the mobility benefits of a dynamic sliding system.

From the daily practice of orthopaedic surgeons came the observation that existing growing spine solutions suffer from the issue of proximal junctional kyphosis (PJK), where a moment in a sagittal plane is applied to the spinal correction system. By providing a connector for slidably coupling to a rod, while substantially preventing any relative rotation in the sagittal plane, the problem of kyphosis is believed to be significantly reduced.

Preferably, the guiding mechanism is arranged to prevent any movement with respect to the rod other than a movement with respect to said longitudinal direction of the rod and preferably axial rotation around said longitudinal direction. Generally, it is preferred if the guiding mechanism is arranged to prevent any rotation of the body with respect to the rod in any plane containing the longitudinal direction or axis of the rod. Rotation in for instance the sagittal plane and the coronal plane is thus prevented.

The coupling mechanism is arranged to couple to another orthopaedic device, for instance the anchoring device as mentioned. The coupling mechanism may also be formed integrally with another constructional element, such as an anchoring device. Rotation of such a device in the sagittal and coronal planes is then prevented. As the coupling mechanism, or another device as mentioned above, is preferably rigidly coupled to the body, also the relative movement of the coupling mechanism, and therewith any coupled other device, is fixed. Also such a device is thus not allowed to rotate in the sagittal or coronal plane, or any other plane containing the longitudinal direction or axis as mentioned above.

As mentioned, the guiding mechanism is arranged to prevent any movement, preferably with respect to the rod, other than a translation along or rotation around the longitudinal direction of the rod. In the alternative, only translation is allowed. The connector is then only allowed to translate longitudinally along the length of the rod. Any rotation of the body in other planes is then prevented with respect to the rod.

It is however preferred if guiding mechanism is further, or alternatively only, arranged to also allow a rotation around said longitudinal direction of the rod. In this way, rotational and axial mobility may be maintained while constraining any other movement, including a movement in a sagittal plane associated with kyphosis. As mentioned, rotation of the body with respect to the rod in other planes, such as the coronal and sagittal planes, is preferably also prevented.

It is remarked that when a curved rod is used, as will be explained in greater detail below, longitudinal direction of the rod may vary along the length, also within the connector. The guiding mechanism is then preferably arranged to prevent any movement other than a movement with respect to an average longitudinal direction, as seen along the length, or to substantially prevent any movement as described. Preferably, the guiding mechanism is arranged to allow relative movement with respect to the rod in only one degree of freedom, preferably sliding of the connector on the rod, i.e. in the longitudinal or axial direction. More preferably, the guiding mechanism is further arranged to further allow movement in a second degree of freedom, i.e. rotation or roll around the rod. Thus, preferably the guiding mechanism is arranged to only allow movement of the body of the connector, and therewith the coupling mechanism, in only two degrees of freedom, i.e. axial sliding along and rotation around the rod in the transverse plane.

According to a preferred embodiment, the body has a first tubular opening and the guiding mechanism is provided in the first tubular opening. The rod may thus be constrained by the geometric properties of the connector.

According to a preferred embodiment, the guiding mechanism has at least two guiding portions provided at a mutual distance in said first tubular opening. The rod may be constrained by aligned guiding portions acting as a constraining conduit for the rod.

Preferably, the at least two guiding portions are provided at the end parts of the first tubular opening. This allows easy insertion of the guiding portions in the body and the physical size of the connector may be kept to a minimum. This further allows the receiving and guiding rods having more pronounced curvatures, as will be further explained below.

According to a preferred embodiment, the first tubular opening has a first internal diameter, the guiding portions have a second internal diameter, and the first internal diameter is larger than the second internal diameter. The first tubular opening may accommodate a rod with a curvature, in particular a rod contoured into the desired shape in both the coronal and sagittal plane.

According to a preferred embodiment, each guiding portion has a bearing or set of bearings and a bearing holder. The bearing portion is preferably a bearing mounted on the bearing holder and allowed to rotate with respect to said holder. Preferably, the bearing is rotatable around a plurality of axis with respect to said holder. Sliding of the rod is allowed at low friction while preventing metal debris and metallosis or metal poisoning, due to its specific lining Curved rods are further efficiently received and guided.

According to a preferred embodiment, the guiding portions are spherical bearings. A rod with a curvature may then easily be accommodated through the ends of the tubular opening, since spherical bearings allow three rotations around roll, yaw and pitch axes.

For efficient fabrication, it is preferred if the guiding portions are slid inside the body through slot openings in the body.

According to a preferred embodiment, the guiding portions are slid inside the body via the ends of the first tubular opening. Bearing holders may thus be inserted in the body while the body may retain its integrity and while space for a locking mechanism in the middle part of the connector may be preserved.

According to a preferred embodiment, the guiding portions are fixed using one of a pin mechanism, a thread mechanism, a bayonet mechanism, a snap fit mechanism. More in particular, the bearing holders are fixed using one of a pin mechanism, a thread mechanism, a bayonet mechanism, a snap fit mechanism. In this way, bearings and their bearing holders may be inserted in the body in a tool less manner According to a preferred embodiment, a spring is preferably provided and preferably there is further a docking provided for said spring, wherein said spring is arranged for providing a distraction force along the longitudinal direction of the rod. In this way, distraction may be implemented in combination with sagittal control. A first end of the spring may abut the body of the connector, for instance at said docking, while a second end of the spring may abut a stop provided on the rod. Preferably, the spring is arranged around the rod.

According to a preferred embodiment, the body has a locking mechanism for locking the rod in a fixed position with respect to the body. In this way, a permanent fixation may be achieved, which is particularity useful when used in combination with a spring while implanting such a system.

According to a preferred embodiment, the coupling mechanism has a second tubular opening for receiving a stationary rod. Such a rod can be used for fixation to at least one, preferably a plurality of vertebrae, for instance using bone pins. Preferably, said second tubular opening has a direction substantially parallel to the direction of the first tubular opening. In this way, the transmission of a torque between the rods may be minimized.

According to a preferred embodiment, the coupling mechanism has a locking mechanism for locking the stationary rod with respect to the coupling mechanism. In this way, a kyphosis moment on the stationary rod may be transmitted to the connector.

Another object, among other objects, is met by a connector assembly for spinal correction comprising a connector as described above and an anchoring device for anchoring to the spine, said anchoring device being coupled to the connector via the coupling mechanism of the connector. In this way, kyphosis of a part of the spine may be controlled.

According to a preferred embodiment, the anchoring device has a stationary rod. In particular, the anchoring device has one or more anchoring elements for anchoring in one or more vertebrae. More in particular, the stationary rod is fixed by two anchoring elements in two neighbouring vertebrae. In this way, a kyphosis force on a part, mostly a superior part, of the spine may be controlled.

Another object, among other objects, is met by a spinal correction system comprising a rod for spinal correction and at least one connector according to any of the above preferred embodiments. In this way, a complete system for growth and/or distraction systems controlling kyphosis is provided.

According to a preferred embodiment, the system further comprises an anchoring device to form with the at least one connector a connector assembly according to any of the above embodiments. In particular, one or more anchors are further provided for anchoring the rod in the spine. In this way, kyphosis of a part, mostly a superior part, of the spine may be controlled.

According to another aspect, is provided a kit of parts comprising at least two of a connector, an anchoring device and a rod.

Another object, among other objects, is met by a method for providing a spinal correction to a patient, comprising the steps of providing a rod for attachment to the spine of the patient, providing an anchoring device for attachment to the spine of the patient, providing a connector according to any of the above preferred embodiments for connection to the rod and the anchoring device, and coupling the connector to the rod. According to a preferred embodiment, the method further comprises coupling the connector to the anchoring device.

The method preferably comprises a step of attaching the rod to the spine, for instance using bone screws or bone pins. Providing the anchoring device may comprise attaching the anchoring device to the spine, in particular to at least one vertebra. The anchoring device may be unitary with the connector, in the alternative the connector can be coupled to the anchoring device.

The connector is preferably coupled to the rod by sliding the connector, preferably using the guiding mechanism as described above, over the rod. After connection to the anchoring device, the anchoring device can be attached to the spine. Or the connector can be attached to the anchoring device once the anchoring device is attached to the spine.

In particular the connector or system as described may be used for the following non exhaustive list of spinal applications:
as a “soft landing” in long trajectory segmental fusions, for example in degenerative scoliosis. In that application an additional stopper ring can be used at the end of the rod without interposition of a small absorption tube or spring;
to guide spinal stiffening in degenerative disc disease to prevent or treat degenerative scoliosis;
to allow load sharing of the axial forces between instrumentation and the vertebrae in long trajectory stabilizations, this will prevent disuse osteoporosis;
to maintain mobility between instrumented segments which do not have to fuse, but need to support sagittal stability, for example, but not limited to, in fracture healing;
to allow passive growth in growth guiding systems. Due to the option to combine several vertebrae and or lamina and ribs, the anchors can be adapted to the specific needs and are much stronger;
to allow active growth/distraction in hybrid growing rod constructs such as in combination with Magnetically Controlled Growing Rods (MCGR);
in combination with a stop ring at the apical side of the connector. In such a way the device allows to maintain rotational mobility in active growth or supporting systems like traditional growing rods and MCGR;
the stop ring at the end of the rod can prevent the rod coming loose from the connector.

Thus, according to a further aspect, a method is provided according to any of the above applications, including the step of providing a connector or system as mentioned above.

Although arranged for spinal correction, the connector could be arranged for other orthopaedic applications related to bones or joints in general. For instance, the connector could be used for slidably connecting to rods in system connected to hands and feet. The principle of the connector described here is therefore not limited to spinal correction insofar as the concept of an orthopaedic connector constraining a moment in a given plane as disclosed here may also be declined accordingly for other types of orthopaedic applications.

It is noted that various known planes and directions will be referred to in the present application and are here explained for the ease of the reading. A sagittal plane refers to a plane defined by two axes, one drawn between a head (superior) and tail (inferior) of the body, and one drawn between a back (posterior) and front (anterior) of the body. A coronal plane refers to a plane defined by two axes, one drawn between a center (medial) to side (lateral) of the body, and one drawn between a head (superior) and tail (inferior) of the body. A transverse plane refers to a plane defined by two axes, one drawn between a back and a front of the body, and one drawn between a center and a side of the body.

This and other aspects of the present invention will now be described in more detail, with reference to the appended drawings showing currently preferred embodiments of the invention, wherein:

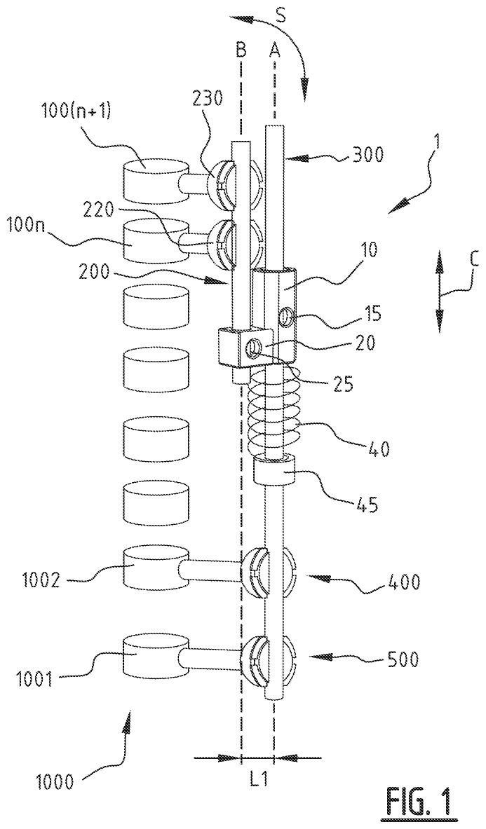

FIG. 1 illustrate a view of a complete spinal correction system comprising the connector;

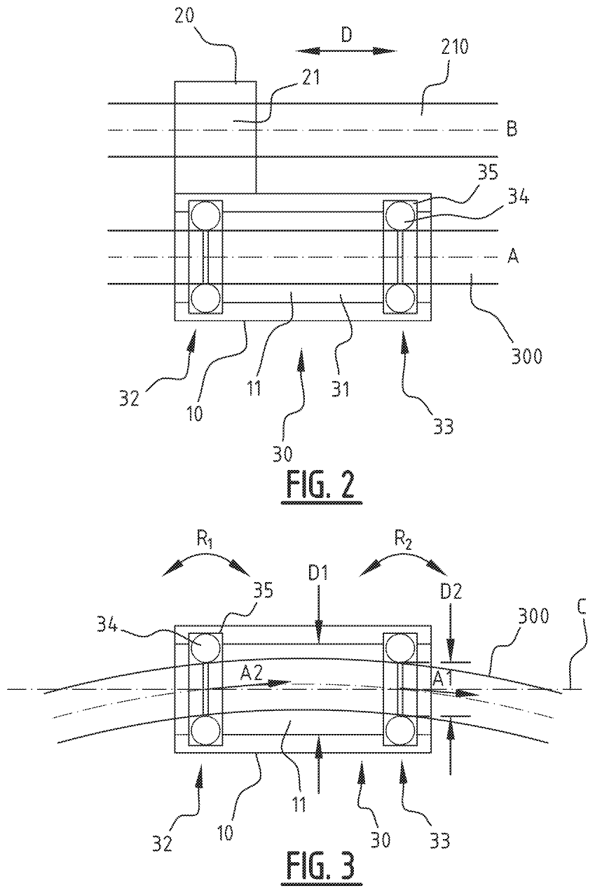

FIG. 2 illustrates a cross-section of the connector according to an embodiment with a straight rod;

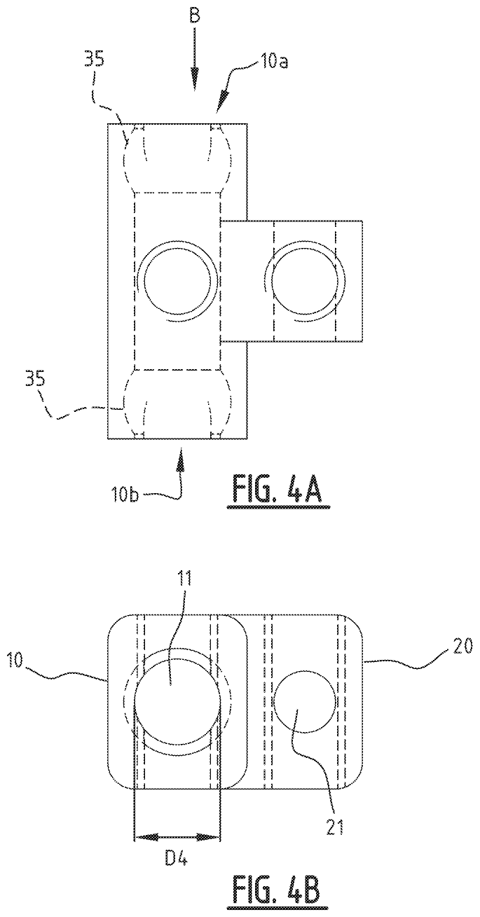

FIG. 3 illustrates a cross-section of the connector according to an embodiment with a rod having a curvature;

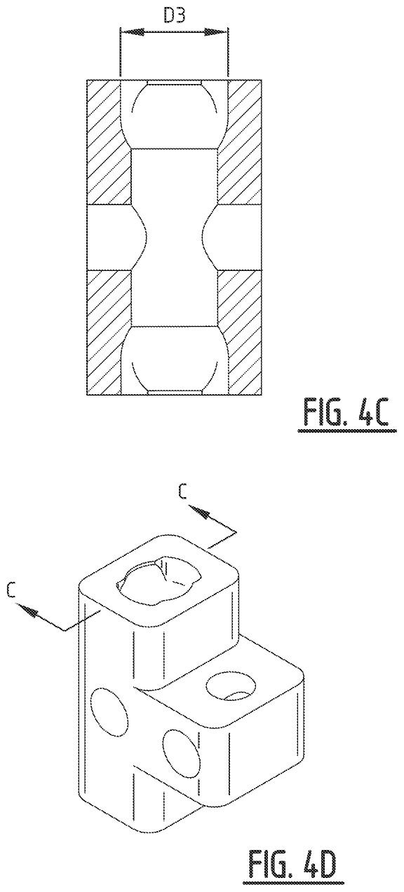

FIG. 4 illustrates a perspective view and three cross-sections of the connector according to an alternative embodiment with a connector body having spherical bearing holders;

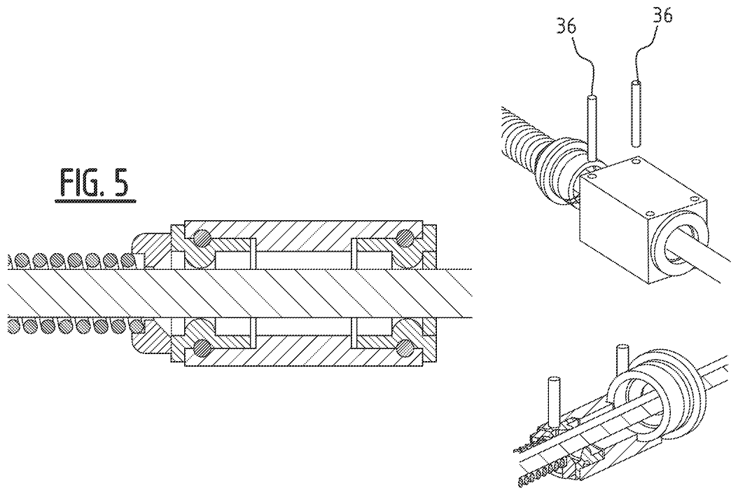

FIG. 5 illustrates a cross-section of the connector according to an alternative embodiment with a connector body configured having a pin mechanism for locking bearing holders;

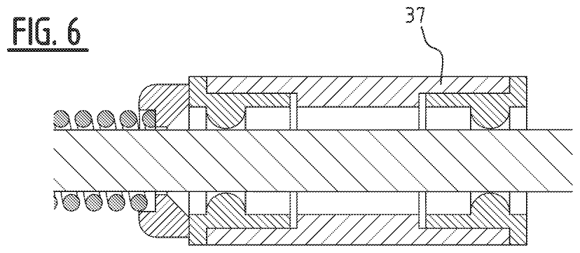

FIG. 6 illustrates a cross-section of the connector according to an alternative embodiment with a connector body configured having a thread mechanism for locking bearing holders;

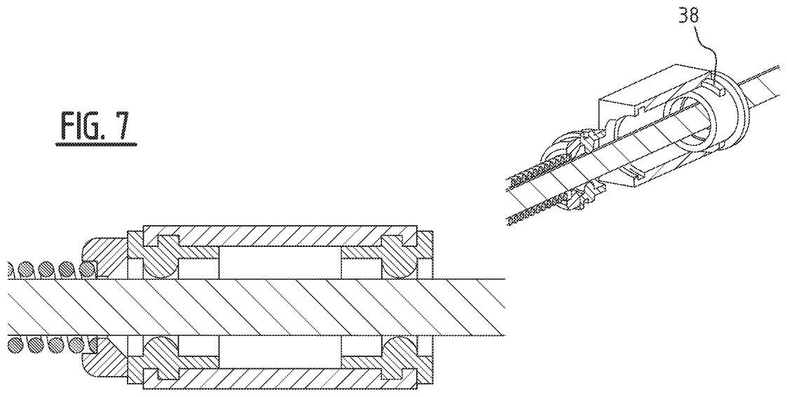

FIG. 7 illustrates a cross-section of the connector according to an alternative embodiment with a connector body configured having a bayonet mechanism for locking bearing holders;

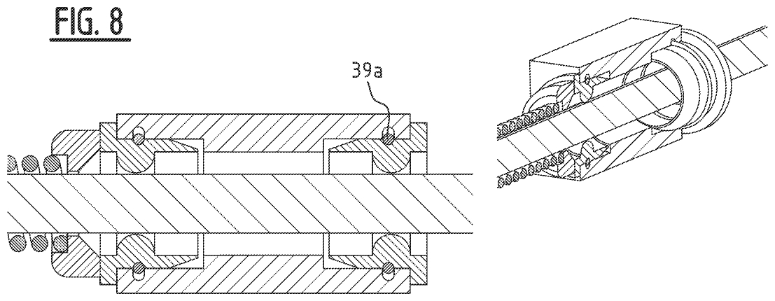

FIG. 8 illustrates a cross-section of the connector according to an alternative embodiment with a connector body provided with a locking ring mechanism for locking bearing holders;

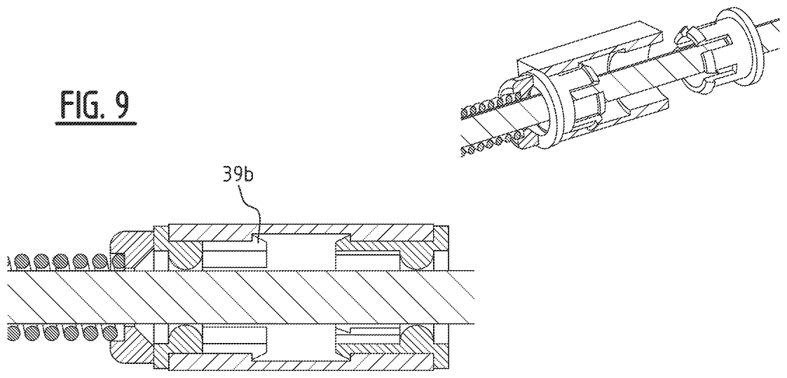

FIG. 9 illustrates a cross-section of the connector according to an alternative embodiment with a connector body having a snap fit mechanism for locking bearing holders;

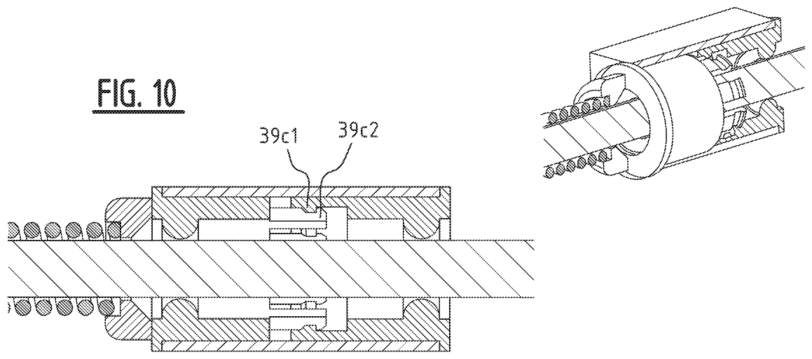

FIG. 10 illustrates a cross-section of the connector according to an alternative embodiment with a connector body configured for interlocking bearing holders.

FIG. 1 shows a spinal correction system 1 comprising a connector 100, an anchoring device 200, a rod 300 and anchoring elements 400 and 500 for anchoring to the spine 1000. Each element of the system will now be described successively.

As shown in FIG. 1, the rod 300 is an elongate member, having a cylindrical shape and being secured to the spinal column 1000 via two anchoring elements 500, 600. The anchoring elements 500 600 are mounted, or fixed to two vertebrae 1001, 1002 located at a first position along the spine 1000, in an inferior part of the spinal column 1000. Optionally the rod 300 may be anchored at additional anchoring points. The anchoring elements 400, 500 are further adapted to receive and secure the rod 300, such that the rod 300 is made solidary with the two vertebrae 1001 and 1002.

The rod 300 in FIG. 1 is further illustrated as having a round cross section with a diameter D1. Yet other embodiments may be envisaged with a rod having a different contour and in particular with a rod having one or more flat portions. As illustrated in FIG. 1, the rod 300 is a straight rod having an axis of symmetry. In such a case, a longitudinal direction A of the rod 300 coincides with the longitudinal axis of the rod 300.

The anchoring device 200 comprises a stationary rod 210 extending along a longitudinal direction B and two anchoring elements 220 and 230. The anchoring elements 220 and 230 may be similar to the anchoring elements 400 and 500 and may be mounted or fixed to two vertebrae 100n and 100(n+1) located at a second position along the spine 1000, in a superior part of the spinal column 1000. The second position is in this example located above the first position along the spinal column 1000. Optionally the stationary rod 210 may be anchored at additional anchoring points.

The connector 100 comprises a body 10, a coupling mechanism 20 for coupling the body 10 to the anchoring device 200, and a guiding mechanism 30. The guiding mechanism 30 (see also FIGS. 2a and 2b) is configured to guide the rod 300 in a sliding movement along the longitudinal direction A of the rod 300, and is further arranged to prevent any movement with respect to the rod other than a movement with respect to said longitudinal direction A of the rod 300.

The body 10 illustrated in FIG. 1 has an elongated tubular shape with a longitudinal direction (see arrow C in FIG. 1) matching the longitudinal direction A of the axis of the rod 300, such that the rod 300 may cross right through the body 10 along the longitudinal direction A. Although represented as rather a tubular shaped element, other shapes of a body may be envisaged for receiving the rod 300 in the body 10.

The connector 100 is such that when assembled in the system of FIG. 1 any movement in a sagittal plane (see arrow S in FIG. 1 illustrating this movement) may be constrained. When the rod 300 is mounted in the connector 100, or in other words, when the connector 100 is coupled to the rod 300, the connector 100 may slide freely along the longitudinal direction A of the rod 300 while any other movement is prevented. In particular any movement in a sagittal plane is prevented. Only a translation along the direction A and a roll rotation movement around the axis of the rod are allowed by the guiding mechanism 30 of the connector 100. In this way, rotational and axial mobility for growth or distraction is maintained while preventing the issue of kyphosis, particularly present in cases of growing or distraction systems.

For the option where some distraction between the lower vertebrae 1001, 1002 and the upper vertebrae 100n, 100(n+1) is desired, a spring mechanism 40 may be provided around the rod 300 for constraining the translation movement of the connector 100 along the rod 300 by exerting a compression effort between a stopper element 45 and the body 10 of the connector 100. The stopper element can be used proximal and distal depending on the desired function.

In the embodiment of FIG. 1, the coupling mechanism 20 is integrally attached to the body 10 to form an L-shaped connector 100. The long side of the L-shape corresponds substantially to the body 10 of the connector 100 and extends in the longitudinal direction A of the rod 300. The short side of the L-shaped connector 100 corresponds substantially to the coupling mechanism 20. Alternatively the coupling mechanism 20 may provide coupling to an additional element (not represented in any of the figures) configured solely for anchoring and/or to receive and secure the stationary rod 210 or any other orthopaedic device.

The coupling mechanism 20 is configured to receive and secure the stationary rod 210, such that the stationary rod 210 is made solidary with the coupling mechanism 20 and thus with the connector 100. The coupling mechanism 20 is configured to secure the stationary rod 210 in a direction B. The direction B may be substantially parallel to the direction A such that the rod 300 and the rod 210 are substantially parallel to each other and at a mutual lateral distance L1 chosen to minimize the transmission of a torque between the rods 210 and 300. The rod 300 is further positioned next to the anchoring device 200 to preserve mobile segments of the spine as much as possible

The coupling mechanism 20 may comprise a locking mechanism 25 for locking the stationary rod 210 with respect to the coupling mechanism 20. A screw may for instance be provided in an opening in the coupling mechanism 20 for securing the stationary rod 210 to the coupling mechanism 20.

Similarly the body 10 may comprise a locking mechanism 15 for locking the rod 300 with respect to the body 10. A screw may for instance be provided in an opening in the body 10 for securing the rod 300 to the body 10.

FIG. 2a illustrates a cross-section of the connector 100 connected to a straight rod 300. In particular FIG. 2a shows how the body 10 has a first tubular opening 11 extending through the body 10 in a longitudinal direction of the body 10 (coinciding with direction A in FIG. 2a), creating a receptacle for the rod 300. The guiding mechanism 30 is then provided in the first tubular opening 11 for guiding the rod 300 in a sliding movement along the longitudinal direction A of the rod 300.

As illustrated the guiding mechanism 30 provided inside the tubular opening 11 has at least two guiding portions 32, 33 provided at a mutual distance D in said first tubular opening 11. In particular the two guiding portions 32, 33 are provided at or near the end parts of the first tubular opening 11. Each guiding portion 32, 33 comprises an annular bearing 34 housed in a bearing holder 35. Each annular bearing 34 can rotate in its bearing holder such that the rod 300 may slide and roll with respect to the direction A. The bearings 34 may be spherical bearings.

The cross section of the coupling mechanism 20 shows further a second tubular opening 21 in the coupling mechanism 20 for receiving the stationary rod 210. The second tubular opening 21 has a longitudinal direction B substantially parallel to the longitudinal direction of the first tubular opening 11.

FIG. 2b illustrates a cross-section of the connector when connected to a rod 300 having a curvature. Of significance is that the bearings 34 are spherical bearings allowing a rotation according to a roll, pitch and yaw rotation axis, see arrows R1 and R2 in FIG. 2b, allowing the bearings to adjust to the surface of the curved rod 300. The first internal diameter D1 of the tubular opening 11 is larger than the second internal diameter D2 of the bearings 34 to allow the movement of the rod having a curvature in the region there between. The second internal diameter D2 is dimensioned relative to the diameter of the rod 300 to allow a translation with a predetermined friction. In this case the sliding movement of the rod is guided locally at each guiding portion 32, respectively 33 in a longitudinal direction A1, respectively A2. It is noted that the longitudinal directions A1 and A2 of the sliding movement of the rod at the guiding portions 32 and 33 are substantially the same as the longitudinal direction C of the first tubular opening 11.

FIG. 3 illustrates a perspective view and three cross-sections of a connector 100 with a connector body 10 comprising two bearing holders 35 formed as spherical cavities inside the tubular opening 11 in the body 10. At the extremities 10a and 10b of body 10 along the longitudinal direction A, the tubular opening 11 mouths into an oval shape having a long axis dimension D3 greater than a short side dimension D4, wherein D3 and D4 are chosen relative to the external diameter of the bearings 34, such that D4 is greater than the external diameter of the bearings 34 and D4 is smaller than the external diameter of the bearings 34. The oval shaped holes at the ends 10a and 10b of the body allow the insertion and the shape locking of annular bearings 34 in the spherical bearing holder cavities 35.

FIGS. 4-10 illustrate alternative embodiments where the bearing holders 35 are separate elements form the body 10. In particular the embodiments of FIGS. 4-10 show alternatives where the bearings 34 and their bearings holders 35 are slid inside the body 10 via the ends of the first tubular opening 11.

FIG. 4 illustrates a cross-section of the connector according to an alternative embodiment of the invention with a connector body configured having a pin mechanism 36 for locking bearing holders 35. First the bearing holders 35 housing the bearings 34 are slid from each end of the body 10 inside the tubular opening 11, then pins 36 are engaged from the outside of the housing into holes provided in the body 10 and the bearing holders 35 to lock the bearing holders 35 inside the body 10.

FIG. 5 illustrates a cross-section of a connector with a connector body 10 configured having a thread mechanism 37 for locking bearing holders 35. Each bearing holder 35 is provided with a thread 37 mating a thread provided on the inner side of the body 10 such that the bearing holders and the bearings 34 may be presented via the ends and fixed by rotation inside the body 10.

FIG. 6 illustrates a cross-section of the connector with a connector body 10 configured having a bayonet mechanism 38 for locking bearing holders 35. Each bearing holder 35 is provided with a bayonet 38 mating a bayonet thread provided on the inner side of the body 10 such that the bearing holders 35 and the bearings 34 may be presented via the ends and fixed by rotation inside the body 10.

FIG. 7 illustrates a cross-section of the connector with a connector body 10 provided with a locking ring mechanism 38a for locking bearing holders 35. Each bearing holder 35 is provided with a locking ring 38a meant to lock into a recess provided on the inner side of the body 10 such that the bearing holders 35 and the bearings 34 may be presented via the ends and snap-fitted when the locking ring 38a falls into the recess inside the body 10.

FIG. 9 illustrates a cross-section of the connector with a connector body 10 having a snap fit mechanism for locking bearing holders 35. Each bearing holder 35 is provided with a locking profile 38b for a snap fit with a complementary locking profile provided on the inner side of the body 10 such that the bearing holders 35 and the bearings 34 may be presented via the ends and snap fitted inside the body 10 when the locking profiles 38b on the bearing holder 35 and the body interlock.

FIG. 9 illustrates a cross-section of the connector with a connector body 10 configured for interlocking bearing holders 35. Bearing holders 35 on opposite ends of the body are configured to interlock inside the body via interlocking profiles 39c such that the bearing holders 35 and the bearings 34 may be presented via the ends and snap fitted together inside the body 10 when the locking profiles 39c of the bearing holders 35 interlock.

It is further noted that the above list of ways of mounting the bearings 34 inside the connector 10 is not exhaustive. Other alternatives may be envisaged by a skilled person depending on the circumstances and materials used. One can imagine for instance alternatives where the bearings would not be inserted by the ends of the tubular opening 11 but for instance via lateral openings and slots in the body 10.

As far as materials are concerned, the connector is preferably made of titanium and the rod of cobal-chromium (CoCr) to prevent titanium-on titanium friction and metal debris. Other materials suitable, such as for instance poly ethylene, for the above described movements may however be envisaged as well.

According to a further embodiment a method for spinal correction is provided, comprising the steps of:
Providing a rod 300 for attachment to the spine,
Providing an anchoring device 200 for attachment to the spine
Providing a connector 100 according to the invention for connection to the rod 300 and the anchoring device 200, and
Further comprising the step of coupling the connector 100 to the rod 300.

A further step comprises coupling the connector (100) to the anchoring device (200).

The method preferably comprises a step of attaching the rod 300 to the spine 1000, for instance using anchoring elements 400 and 500, being bone screws or bone pins. Providing the anchoring device 200 may comprise attaching the anchoring device 200 to the spine 1000, in particular to at least one vertebra 100n and optionally to another vertebrae 100(n+1). The anchoring device 200 may be unitary with the connector 100, in the alternative the connector 100 can be coupled to the anchoring device 200.

The connector 100 is preferably coupled to the rod 300 by sliding the connector 100, preferably using the guiding mechanism 30 as described above, over the rod 300. After connection to the anchoring device 200, the anchoring device 200 can be attached to the spine.

Alternatively the connector 100 can be attached to the anchoring device 200 once the anchoring device 200 is attached to the spine.

Surgery can be performed in particular through a posterior midline skin incision. The anchoring elements 400, 500, 220, 230 can be placed with the freehand technique. The rods 200 and 300 can be passed sub-fascially and contoured into the desired shape in both the coronal and sagittal plane.

It is noted that a surgeon may decide depending on circumstances to alter the sequence of the steps, and start with any one of the anchoring device, the rod and or the connector.

The present invention is further illustrated by the following embodiments, which are not intended to limit the scope of the invention in any way:
1. Connector (100) for slidably connecting an anchoring device (200) to a rod (300) in a spinal correction system, comprising
   a body (10),
   a coupling mechanism (20) for coupling the body (10) to the anchoring device (200),
   a guiding mechanism (30) arranged to guide the rod (300) in a sliding movement along a longitudinal direction (A) of the rod (300), wherein said guiding mechanism is further arranged to prevent any movement, with respect to the rod (300), other than a translation along the longitudinal direction (A) of the rod (300) and a rotation around said longitudinal direction (A) of the rod (300).
2. Connector according to embodiment 1, wherein the guiding mechanism is arranged to prevent any rotation of the body with respect to the rod in any plane containing the longitudinal direction or axis of the rod.
3. Connector according to any of the above embodiments, wherein the body (10) has a first tubular opening (11), and wherein the guiding mechanism (30) is provided in said first tubular opening (11).
4. Connector according to the previous embodiment, wherein the guiding mechanism (30) has at least two guiding portions (32, 33) provided at a mutual distance (d) in said first tubular opening (11).
5. Connector according to the previous embodiment, wherein the at least two guiding portions (32, 33) are provided at the end parts of the first tubular opening (11).
6. Connector according to any one of embodiments 3-5, wherein the first tubular opening (11) has a first internal diameter (D1), the guiding portions (32, 33) have a second internal diameter (D2), and the first internal diameter (D1) is larger than the second internal diameter (D2).
7. Connector according to any one of embodiments 4-6, wherein each guiding portion (32, 33) has a bearing (34) and a bearing holder (35).
8. Connector according to any one of embodiments 4-7, wherein the guiding portions (32, 33) are spherical bearings.
9. Connector according to any one of embodiments 4-8, wherein the guiding portions (32, 33) are slid inside the body 10 through slot openings (12, 13) in the body (10).
10. Connector according to any one of embodiment 4-8, wherein the guiding portions (32, 33) are slid inside the body (10) via the ends of the first tubular opening (11).
11. Connector according to the previous embodiment, wherein the guiding portions (32, 33) are fixed using one of a pin mechanism (36), a thread mechanism (37), a bayonet mechanism (38), a snap fit mechanism (39).
12. Connector according to any one of embodiments 7-11, wherein the bearing holders (32, 33) are fixed using one of a pin mechanism (35), a thread mechanism (36), a bayonet mechanism (37), a snap fit mechanism (38).
13. Connector according to any of the above embodiments, further comprising a spring (40) for constraining the translation of the rod (300) along its longitudinal direction (A).
14. Connector according to any of the above embodiments, wherein the body (10) has a locking mechanism (15) for locking the rod in a fixed position with respect to the body (10).
15. Connector according to any of the above embodiments, wherein the coupling mechanism (20) has a second tubular opening (21) for receiving a stationary rod (210), said second tubular opening (21) having an direction (B) substantially parallel to the direction of the first tubular opening (11).
16. Connector according to the previous embodiment, wherein the coupling mechanism (20) has a locking mechanism (25) for locking the stationary rod (210) with respect to the coupling mechanism (20).
17. Connector assembly (100,200) for spinal correction comprising a connector (100) according to any of the above embodiments and an anchoring device (200) for anchoring to the spine, said anchoring device (200) being coupled to the connector (100) via the coupling mechanism (20) of the connector.
18. Connector assembly according to the previous embodiment, wherein the anchoring device (200) has a stationary rod (210).
19. Connector assembly according to embodiment 17 or 18, wherein the anchoring device (200) has one or more anchoring elements (220,230) for anchoring in one or more vertebrae.
20. Connector assembly connector according to embodiment 18 or 19, wherein the stationary rod (210) is fixed by two anchoring elements (220, 230) in two neighboring vertebrae.
21. Spinal correction system comprising a rod (300) for spinal correction and at least one connector (100) according to any of embodiments 1-16.
22. System according to the previous embodiment further comprising an anchoring device (200) to form with the at least one connector (100) a connector assembly (100, 200) according to any one of the embodiments 17-20.
23. System according to the previous embodiment, further comprising one or more anchors (400, 500) for anchoring the rod (300) in the spine.
24. Kit of parts comprising at least two of a connector (100), an anchoring device (200), and a rod (300) according to any of the preceding embodiments.
25. Method for providing a spinal correction to a patient, comprising the steps of:
   providing a rod (300) for attachment to the spine of the patient;
   providing an anchoring device (200) for attachment to the spine of the patient;
   providing a connector (100) according to any of embodiments 1-16 for connection to the rod (300) and the anchoring device (200);
   coupling the connector (100) to the rod (300).
26. Method according to embodiment 25, further comprising coupling the connector (100) to the anchoring device (200).

Whilst the principles of the invention have been set out above in connection with specific embodiments, it is understood that this description is merely made by way of example and not as a limitation of the scope of protection which is determined by the appended claims.

1. A connector for slidably connecting an anchoring device to a rod in a spinal correction system, comprising
a body, wherein the body has a first tubular opening,
a coupling mechanism for coupling the body to the anchoring device,
a guiding mechanism provided in said first tubular opening (11) and arranged to guide the rod in a sliding movement along a longitudinal direction of the rod, wherein said guiding mechanism has at least two guiding portions provided at a mutual distance in said first tubular opening and is further arranged to prevent any movement, with respect to the rod, other than a translation along the longitudinal direction of the rod and a rotation around said longitudinal direction of the rod.

2. The connector according to claim 1, wherein the guiding mechanism is arranged to prevent any rotation of the body with respect to the rod in any plane containing the longitudinal direction or axis of the rod.

3. The connector according to claim 1, wherein the at least two guiding portions are provided at the end parts of the first tubular opening.

4. The connector according to claim 1, wherein the first tubular opening has a first internal diameter, the guiding portions have a second internal diameter, and the first internal diameter is larger than the second internal diameter.

5. The connector according to claim 1, wherein each guiding portion has a bearing and a bearing holder.

6. The connector according to claim 1, wherein the guiding portions are spherical bearings.

7. The connector according to claim 1, wherein the guiding portions are slid inside the body through slot openings in the body.

8. The connector according to claim 1, wherein the guiding portions are slid inside the body via the ends of the first tubular opening.

9. The connector according to claim 8, wherein the guiding portions are fixed using one of a pin mechanism, a thread mechanism, a bayonet mechanism, and a snap fit mechanism.

10. The connector according to claim 5, wherein the bearing holders are fixed using one of a pin mechanism, a thread mechanism, a bayonet mechanism, and a snap fit mechanism.

11. The connector according to claim 1, further comprising a spring for constraining the translation of the rod along its longitudinal direction.

12. The connector according to claim 1, wherein the body has a locking mechanism for locking the rod in a fixed position with respect to the body.

13. The connector according to claim 1, wherein the coupling mechanism has a second tubular opening for receiving a stationary rod, said second tubular opening having a direction substantially parallel to the direction of the first tubular opening.

14. The connector according to claim 13, wherein the coupling mechanism has a locking mechanism for locking the stationary rod with respect to the coupling mechanism.

15. A connector assembly for spinal correction comprising:
the connector according to claim 1, and
an anchoring device for anchoring to the spine, said anchoring device being coupled to the connector via the coupling mechanism of the connector.

16. The connector assembly according to claim 15, wherein the anchoring device has a stationary rod.

17. The connector assembly according to claim 15, wherein the anchoring device has one or more anchoring elements for anchoring in one or more vertebrae.

18. The connector assembly according to claim 16, wherein the stationary rod is fixed by two anchoring elements in two neighboring vertebrae.

19. A spinal correction system comprising the rod for spinal correction and at least one said connector according to claim 1.

20. The spinal correction system according to claim 19, further comprising an anchoring device to form a connector assembly with the at least one connector, said anchoring device being coupled to the connector via the coupling mechanism of the connector.

21. The spinal correction system according to claim 20, further comprising one or more anchors for anchoring the rod in the spine.

22. A kit of parts comprising at least two of a connector, an anchoring device, and a rod, according to claim 1.

23. A method for providing a spinal correction to a patient, comprising the steps of:
providing a rod for attachment to the spine of the patient;
providing an anchoring device for attachment to the spine of the patient;
providing the connector according to claim 1 for connection to the rod and the anchoring device; and
coupling the connector to the rod.

24. The method according to claim 23, further comprising coupling the connector to the anchoring device.
